(19) United States
(12) Patent Application Publication
(10) Pub. No.: US 2007/0275030 A1
(43) Pub. Date: Nov. 29, 2007

(54) ANTI-CROSS-LINKING AGENTS AND METHODS FOR INHIBITING CROSS-LINKING OF INJECTABLE HYDROGEL FORMULATIONS

(75) Inventors: Orhun K. MURATOGLU, Cambridge, MA (US); Ebru Oral, Charlestown, MA (US); Hatice Bodugoz-Senturk, Boston, MA (US)

Correspondence Address: PROSKAUER ROSE LLP, 1001 PENNSYLVANIA AVE, N.W.,, SUITE 400 SOUTH, WASHINGTON, DC, 20004, US

(73) Assignee: The General Hospital Corporation dba Massachusetts General Hospital, Boston, MA (US)

(21) Appl. No.: 11/754,003

(22) Filed: May 25, 2007

Related U.S. Application Data
(60) Provisional application No. 60/803,177, filed on May 25, 2006.

(51) Int. Cl. A61K 9/14

(52) U.S. Cl. 424/422; 424/486; 424/487

(57) ABSTRACT

The invention relates to cross-link-resistant injectable hydrogel formulations and methods of partially or practically wholly inhibiting injectable hydrogel formulations from cross-linking, for example, during irradiation, using anti-cross-linking agents, which facilitates injectability of the hydrogel formulation. The invention also relates to methods of making the cross-link-resistant, for example, irradiation cross-link resistant, injectable hydrogel formulations, and methods of administering the same in treating a subject in need.

This application claims priority to U.S. provisional application Ser. No. 60/803,177, filed May 25, 2006, the entirety of which is hereby incorporated by reference.

### FIELD OF THE INVENTION

The invention relates to injectable hydrogel formulations and methods of inhibiting or preventing hydrogel formulations from cross-linking, for example, during irradiation, which facilitates injectability of the hydrogel formulation. The invention also relates to methods of making the injectable hydrogel formulations, and methods of administering the same in treating a subject in need.

### BACKGROUND OF THE INVENTION

Hydrogels are three-dimensional, water-swollen structures composed of mainly hydrophilic homopolymers or copolymers, for example, polyvinyl alcohol (PVA), polyacrylamide (PAAm), poly-N-isopropyl acrylamide (PNIPAAm), polyvinyl pyrrolidone (PVP), poly(ethylene-co-vinyl alcohol). PVA-based hydrogels have been disclosed for use in a variety of biomedical applications. (see Hassan & Peppas, Advances in Polymer Science, vol. 153, Springer-Verlag Berlin Heidelberg, 2000, pp. 37-65; Lowman et al. Ed., John Wiley and Sons, 1999. pp. 397-418).

Hydrogels have been used in a variety of biomedical applications, for example, intervertebral disc replacement or disc augmentation, wound care, cartilage replacement, joint replacement, surgical barriers, gastrointestinal devices, drug delivery, cosmetic and reconstructive surgery, and breast implants.

Hydrogel formulations are also known for their use for injection into body cavities in a liquid form to undergo gelation inside the cavity (see Ruberti and Braithwaite: US Publication Nos. 20040092653 and 20040171740).

Lowman et al. (US Publication No. 2004/0220296) describe a gel formulation comprising poly(N-isopropyl acrylamide), which is also injectable in a liquid form. The liquid formulation undergoes a phase transformation to form a solid hydrogel implant in situ at physiological body temperature.

Another gel formulation has been described by Stedronsky et al. (U.S. Pat. No. 6,423,333). Stedronsky et al. utilized a protein based gel and injected as a fluid into a bodily cavity where it formed a solidified gel.

Sawhney (U.S. Pat. No. 6,818,018) discusses injectable hydrogel formulations that, upon injection into a body cavity, undergo physical associations through chelating agents or thermo-reversible transitions, and then chemically cross-link through the incorporation of cross-linking agents.

Hydrogel formulations, for example, PVA based hydrogel formulations, can be cross-linked by irradiation (see for example, Muratoglu et al., U.S. application Ser. No. 10/962,975 (20060079597A1). PVA based hydrogels also can be made by physical associations; by using a cross-linking molecule, by the freeze-thaw technique (CM Hassan and Peppas NA, Advances in Polymer Science, 2000. 153: p. 37-65) or by using a gellant (see Ruberti and Braithwaite: US Publication Nos. 20040092653 and 20040171740). However, there is no mention of what sterilization or other radiation does to the structure of an injectable formulation of a polymer or a polymer blend.

None of the publications described above disclose an injectable hydrogel formulation that can be injected after being irradiated, for example, for the purpose of sterilizing the formulation prior to injecting or administering into a body or body cavity. It is generally known that irradiation causes cross-linking of most polymers, which compromises the injectability of a hydrogel formulation. Therefore, there is a need for development of a method for inhibiting or preventing irradiation induced cross-linking of injectable hydrogel formulations and a cross-link-resistant hydrogel formulation.

Cross-link-resistant injectable hydrogel formulations, and methods of inhibiting or preventing cross-linking, for example, irradiation induced cross-linking, of injectable hydrogel formulations, methods of administering the same and their use in treating a subject in need are disclosed for the first time by the present invention.

### SUMMARY OF THE INVENTION

It is an object of the present invention to provide an injectable hydrogel formulation comprising an anti-cross-linking agent to facilitate injectability of the hydrogel formulation, wherein the anti-cross-linking agent inhibits, reduces, minimizes, attenuates, or prevents cross-linking, for example, irradiation induced cross-linking, of the hydrogel formulation, thereby providing the hydrogel formulation in an injectable form. In other words, the injectability of the hydrogel formulation can be compromised in absence of the anti-cross-linking agent during irradiation, for example.

An aspect of the invention provides injectable hydrogel formulations and methods to make such formulations whose cross-linking is inhibited and/or injectability is enhanced by the addition of an anti-cross-linking agent. For example, an anti-cross-linking agent can be used to prevent, inhibit, reduce, minimize, attenuate, or decrease cross-linking caused by irradiation and other methods that cause cross-linking, such as crystallization, ionic interactions, thermal cross-linking and others.

This invention facilitates the injectability of hydrogel formulations that would otherwise be difficult, compromised or impossible after gamma sterilization, for example. Therefore, the anti-cross-linking agent is pivotal in the development of injectable hydrogel formulations. The use of an anti-cross-linking agent in an implantable hydrogel also can be selective to inhibit or prevent cross-linking in certain parts of the implantable hydrogel during either gamma sterilization or intentional cross-linking of an implantable hydrogel with high radiation doses.

In another aspect, the invention provides cross-link-resistant and sterile injectable hydrogel formulations comprising at least one anti-cross-linking agent, wherein the anti-cross-linking agent is present, for example, during irradiation, and inhibits, prevents, or reduces cross-linking of the hydrogel formulation, thereby providing a cross-link-resistant and sterile injectable form of hydrogel formulation.

Another aspect of the invention provides injectable hydrogel formulations comprising at least one anti-cross-linking agent, wherein the anti-cross-linking agent is present, for example, during irradiation, and inhibits, prevents, or reduces cross-linking of the hydrogel formulation, thereby providing an injectable form of hydrogel formulation.

Another aspect of the invention provides cross-link-resistant injectable hydrogel formulations comprising at least one anti-cross-linking agent that inhibits cross-linking of the hydrogel formulation, which can be compromised in absence of the anti-cross-linking agent, thereby providing an injectable hydrogel formulation.

Another aspect of the invention provides methods of making a cross-link-resistant and sterile, for example, irradiation-cross-link-resistant and sterile, injectable hydrogel formulation comprising: a) providing monomers, polymers or mixtures thereof in a solvent, thereby forming a hydrogel solution; b) optionally gelling the hydrogel solution; c) contacting the hydrogel solution with one or more anti-cross-linking agents, thereby forming a cross-link resistant hydrogel solution; and d) irradiating the cross-link resistant hydrogel solution, thereby forming an irradiation cross-link-resistant and sterile injectable hydrogel formulation. Gelling refers to transitioning towards and/or achieving a semisolid or semirigid form.

Another aspect of the invention provides methods of making a cross-link-resistant, for example, irradiation-cross-link-resistant, injectable hydrogel formulation comprising: a) providing monomers, polymers or mixtures thereof in a solvent, thereby forming a hydrogel solution; b) optionally gelling the hydrogel solution; c) processing the hydrogel solution to modifying at least one of its physical and/or chemical property; d) contacting the processed hydrogel solution with one or more anti-cross-linking agents, thereby forming an irradiation cross-link-resistant hydrogel solution; and e) irradiating the irradiation cross-link-resistant hydrogel solution, thereby forming an irradiation cross-link-resistant injectable hydrogel formulation.

Another aspect of the invention provides methods of making a cross-link-resistant, for example, irradiation-cross-link-resistant, injectable hydrogel formulation comprising: a) providing monomers, polymers or mixtures thereof in a solvent, thereby forming a hydrogel solution; b) adding at least one anti-cross-linking agent to the hydrogel solution, thereby forming an irradiation cross-link-resistant hydrogel solution; and c) irradiating the irradiation cross-link-resistant hydrogel solution, thereby forming an irradiation cross-link-resistant injectable hydrogel formulation.

Another aspect of the invention provides methods of inhibiting the cross-linking of an injectable hydrogel formulation comprising: a) providing monomers, polymers or mixtures thereof in a solvent, thereby forming a hydrogel solution; b) adding at least one anti-cross-linking agent to the hydrogel solution, thereby forming a cross-link-resistant hydrogel solution; and c) irradiating the irradiation cross-link-resistant hydrogel solution, thereby forming an irradiation cross-link-resistant injectable hydrogel formulation.

According to another aspect of the invention, the gelling is obtained with the aid of a gellant, by chemical cross-linking, by thermal cycling, by irradiation, by changing the chemical or physical environment of the hydrogel formulation such as pH, ionic strength, temperature and/or pressure and/or by the application of an electric or magnetic field or a combination thereof. In some aspects and embodiments of the invention, anti-cross-linking agents can be added during irradiation at the gelling step. Gelling can occur in the presence of the anti-cross-linking agents during the irradiation-induced gelation step as disclosed herein. The presence of an anti-cross-linking agent intended to reduce cross-linking during irradiation and/or during the gelling step may or may not unduly affect the cross-linking by other gelation methods known in the art, depending on the parameters selected.

According to another aspect of the invention, the processing of the hydrogel solution in solid or liquid form is done by dehydration, by dehydration and annealing, by irradiation, by changing the chemical or physical environment of the hydrogel solution such as pH, ionic strength, temperature and/or pressure, by mechanical deformation, by the application of a magnetic or electric field or a combination thereof.

According to another aspect of the invention, the hydrogel is in dry or hydrated form when contacted with the anti-cross-linking agent solution.

In an aspect of the invention, the injectable hydrogel formulation is made of a vinyl polymer, such as poly(vinyl alcohol), poly(vinyl pyrrolidone), an acrylamide polymer such as poly(N-isopropyl acrylamide), an acrylic polymer such as poly(acrylic acid), poly(ethylene glycol) methacrylate, a polyolefin such as polyethylene, copolymers such as poly(ethylene-co-vinyl alcohol) or blends thereof.

In another aspect of the invention, the injectable hydrogel formulation is made of a vinyl polymer, such as poly(vinyl alcohol), poly(vinyl pyrrolidone), an acrylamide polymer such as poly(N-isopropyl acrylamide), an acrylic polymer such as poly(acrylic acid), poly(ethylene glycol) methacrylate, a polyolefin such as polyethylene, copolymers such as poly(ethylene-co-vinyl alcohol) or blends thereof, wherein one of the polymers is grafted on another one.

In another aspect of the invention, the anti-cross-linking agent is an antioxidant, a free-radical scavenger, or a combination thereof. Yet, in another aspect of the invention, the anti-cross-linking agent is selected from the group consisting of: ascorbic acids including ester and acetate forms of vitamin C, carotenoid compounds, lipoic acid; vitamins such as Vitamins E, D, and B; glutathione; quinones; quinines; amino acids such as arginine, cysteine, tryptophan; peroxides; citric acids; succinic acids; phytochemicals such as ferulic acid, lycopene, lumenene; enzymes such as superoxide dismutase, catalase and glutathione peroxidase; phenolic compounds such as α-tocopherol; and a combination thereof.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, that the detailed description and the specific examples, while indicating preferred aspects of the invention, are given by way of illustration only, since various changes and modifications within the spirit and scope of the invention will become apparent to those skilled in the art from this detailed description.

These and other aspects of the invention will become apparent to the skilled artisan in view of the teachings contained herein.

The invention is further disclosed and exemplified by reference to the text and drawings that follow.

### BRIEF DESCRIPTION OF THE DRAWINGS

FIG. 1 shows the rate of viscosity change as a function of decreasing temperature (17.5 wt/v % PVA (115,000 g/mol) and 39 wt/v % PEG (400 g/mol)).

FIG. 2 shows anti-cross-linking effect of vitamin C, which is demonstrated by measuring the viscosity of sterilized PVA solutions. The viscosity values for unsterilized samples are shown with empty symbols and those for sterilized samples are shown in full symbols. The values for 16,000 and 61,000 are on the secondary axis on the right.

FIG. 3 shows the effect of vitamin C on the viscosity of unirradiated, 25 and 100 kGy irradiated PVA solutions containing PVA molecular weight of 16,000 g/mol.

FIG. 4 shows the effect of vitamin C on the viscosity of unirradiated, 25 and 100 kGy irradiated PVA solutions containing PVA molecular weight of 115,000 g/mol.

### DETAILED DESCRIPTION OF THE INVENTION

This invention provides injectable hydrogel formulations and methods for inhibiting, preventing, minimizing, attenuating, or reducing cross-linking, for example, irradiation-induced cross-linking, of the injectable hydrogel formulations (for example, PVA-based hydrogel formulations) during irradiation.

Injectable hydrogel formulations, for example, PVA based hydrogel formulations, can be cross-linked by irradiation (see for example, Muratoglu et al., U.S. application Ser. No. 11/419,142, filed May 18, 2006; also published as WO 2006/125082.

The hydrogels described in the prior art can be used as starting hydrogels in the present invention, see for example, U.S. Pat. Nos. 4,663,358, 5,981,826, and 5,705,780, US Published Application Nos. 20040092653 and 20040171740.

In one aspect of the invention, the polymer or hydrogel solution for forming hydrogels can be made by dissolving one or more polymers in one or more solvents. In addition to polymers, this solution may contain monomers, oligomers, salts, or any inorganic or organic compounds. The solid ingredients can be mixed in the dry state before being dissolved in the solvent or solvents. Alternatively, the solid ingredients may be partially dissolved and mixed in the partially dissolved state in the liquid components and/or the solvents. The partially dissolved ingredients can be processed further without further dissolution. Alternatively, they can be completely dissolved in the solvent or solvents.

Hydrogels can be formed by forming physical cross-links with the aid of a gellant (see Ruberti and Braithwaite, US Publication Nos. 20040092653 and 20040171740; Muratoglu et al. WO 2006/132661), or by thermal cycling (for example, freezing and thawing) or by physical or chemical cross-linking with the aid of a cross-linking agent and/or heat treatment and/or irradiation and/or a change in the physical or chemical environment of the hydrogel formulation such as pH, ionic strength, temperature and/or pressure and/or application of a magnetic or electric field, or any combinations of the above treatments.

The injectable hydrogel formulations defined in the present invention can be used in the body to augment any tissue such as cartilage, muscle, breast tissue, nucleus pulposus of the intervertebral disc, other soft tissue, etc., or can be used as an embolization agent. See U.S. Provisional Application Ser. No. 60/687,317, filed Jun. 6, 2005 (published as WO 2006/132661), the entirety of which is hereby incorporated by reference.

Polyethylene glycol (PEG) has been used in hydrogel preparation, for example in combination with PVA, however, the ability of PEG to interfere with cross-linking has not been previously established. PEG, if present in appropriate proportion, can inhibit or prevent cross-linking.

It also has been known that Vitamin C is an antioxidant and acts as a regenerating agent for oxidized and free radical species in the body. Use of Vitamin C as a radioprotecting agent to prevent the oxidation and degradation of biological systems is known. However, its use to prevent, inhibit or reduce cross-linking of polymers, for example, during irradiation, sterilization and the like, and its role as a free radical scavenger has not been previously established. There is no known prior use of vitamin C with hydrogel forming polymers, such as polyvinyl alcohol (PVA).

According to the invention, the injectable hydrogel formulations can be prepared with various concentrations of an anti-cross-linking agent such as an antioxidant and/or a free radical scavenger, for example, vitamin C. Some embodiments provide methods of inhibiting the cross-linking of the hydrogel mixture, for example, during irradiation and/or sterilization, by keeping concentration of the anti-cross-linking agents high, for example, high concentration of an anti-cross-linking agent, and/or by adding another anti-cross-linking agent, such as vitamin-C, to the mixture.

Some embodiments provide methods of inhibiting the cross-linking of the hydrogel formulation during, for example, during irradiation and/or sterilization, by keeping the concentration of the mixture components high where low concentration of the components does not inhibit cross-linking enough to retain the injectability of the hydrogel formulation. These components can be the gellant, and/or anti-cross-linking agent or another component that is not a gellant.

Anti-cross-linking agent can be present during gelation by irradiation in an amount not sufficient to cause undue inhibition of the gelation of the hydrogel formulation. This depends upon the concentration of the anti-cross-linking agent and the dose rate, and overall dose of irradiation. If the concentration of anti-cross-linking agent is too high or the irradiation dose rate or total dosage is too low, cross-linking of the formulation cannot occur, which will affect the gelation process. Such parameters can be readily determined by the skilled person in view of the teachings contained herein.

In contrast, anti-cross-linking agent can inhibit cross-linking to a sufficient degree that a hydrogel formulation can be injected. This depends upon the concentration of the anti-cross-linking agent and the dose rate, and overall dose of irradiation. If the concentration of anti-cross-linking agent is too low or the irradiation dose rate or total dosage is too high, cross-linking of the formulation can occur, which will affect injectability. Such parameters also can be readily determined by the skilled person in view of the teachings contained herein.

According to an aspect of the invention, an injectable hydrogel formulation comprises at least one anti-cross-linking agent, wherein the anti-cross-linking agent is present, for example, during irradiation and/or sterilization, and prevents, inhibits, minimizes, attenuates, or reduces cross-linking of the hydrogel caused by the radiation, thereby providing a cross-link-resistant injectable form of hydrogel, wherein the anti-cross-linking agent is not a gellant for vinyl polymers such as PVA. Although PEG is known as a gellant for vinyl polymers, according to the invention, PEG can be used to inhibit or prevent cross-linking.

According to an aspect of the invention, an injectable hydrogel formulation comprises at least one anti-cross-linking agent, wherein the anti-cross-linking agent is present, for example, during irradiation or sterilization, and prevents, inhibits, minimizes, attenuates, or reduces cross-linking of the hydrogel, for example, caused by the radiation, thereby providing an irradiation cross-link-resistant injectable form of hydrogel, wherein the anti-cross-linking agent is not a gellant for vinyl polymers such as PVA. Although PEG is known as a gellant for vinyl polymers, according to the invention, PEG can be used to inhibit or prevent cross-linking at some concentration. For example, the concentration at which PEG will act as anti-cross-linking agent depends on the concentration of PVA and the molecular weight of the components (both PVA and PEG). For example, a 17.5 w/v % PVA solution made with PVA of 115,000 g/mol, PEG600 forms a strong gel at about 17.5 w/v %, PEG400 forms a strong gel at about 35 wt/v % and PEG 200 does not form a strong gel below about 50 wt/v % before sterilization. PEG may act as an anti-cross-linking agent at a lower or similar concentration then that at which it forms a strong gel.

According to an aspect of the invention, hydrogel formulations, for example, an injectable PVA-hydrogel formulation, at least one anti-cross-linking agent(s), and optionally PEG, and solvent mixture are prepared in a syringe at an elevated temperature, for example, above 70° C., preferably about 90 to about 95° C. Upon cooling down to below the solidifying temperature or to about room temperature, the mixture forms a hydrogel in the syringe. The solution can be cooled down to about 0° C. or to below 0° C. and maintained for any given time before heating back to about room temperature or to about body temperature or about or above melting temperature of the gel. The syringe is irradiated and/or sterilized in this state. Subsequently, the irradiated and/or sterilized syringe is heated to a temperature to either soften or dissolve the hydrogel or hydrogel formulation to make the mixture injectable and used in the operating room. However, when the sterilization is carried out with ionizing radiation, the hydrogel undergoes varying degrees of cross-linking depending on the concentration of anti-cross-linking agent(s) and/or PEG. For example, at lower PEG concentrations, PVA cross-linking is higher and as a result heating does not liquefy the mixture and injectability of the hydrogel formulation is compromised.

According to another aspect of the invention, a polymer, such as PVA, is dissolved in hydrophilic solvents at various concentrations at various temperatures. Depending on the procedure used to prepare and store the polymeric solutions, the polymer forms physically entangled films, or physically cross-linked crystalline structure with pores. Physically cross-linked structures are dissolved back into solution when the temperature is raised above the temperature where the energy of the physical entanglements and hydrogen bonds that hold the crystals together are exceeded by the kinetic energy of the chains. Alternatively, the formulation may become a solution when the hydrogen bonds are broken at a temperature higher than the lower critical solution temperature such as for NIPAAm-based gels. When hydrogel solutions for forming hydrogels, such as a PVA-hydrogel solution, are irradiated by ionizing irradiation, chemical cross-links are formed between chains with the aid of solvent, which acts as a chain transfer agent for free radicals. These chemically cross-linked structures form a network and are not soluble or do not flow completely when the temperature is raised or lowered.

The term “solvent” refers to what is known in the art as a medium or a combination of media in which vinyl polymers such as poly(vinyl alcohol), acrylamide polymer such as poly(N-isopropyl acrylamide), acrylic polymer such as poly(acrylic acid), poly(ethylene glycol) methacrylate, and polyolefin such as polyethylene or copolymers or blends thereof are soluble. Solvents can be water, and aqueous solutions with additives such as salts, emulsifiers, pH regulators, viscosity modifiers, alcohols, and DMSO, or mixtures thereof or any other mixture that can dissolve the polymer.

According to an aspect of the invention, the polymer solution is made with a solvent or a combination of solvents that dissolve the monomer and/or polymer and/or the anti-cross-linking agent. The polymer solution is then irradiated, thereby forming an injectable hydrogel formulation, which is suitable for in vivo use because it is sterilized and/or the hydrogel formulation is prepared with or the formulation is exchanged with a biocompatible solvent. The injectable hydrogel formulations or compositions and the solvent therein are biocompatible and are made suitable for in vivo use.

According to an aspect of the invention, the polymer solution is made with a solvent or a combination of solvents that dissolve the monomer and/or polymer. The polymer solution is then solidified or gelled by changing the physical or chemical environment of the polymer solution such as pH, ionic strength, pressure and/or temperature. According to one aspect of the invention, the polymer solution is gelled by cooling or heating to below or above its solidification temperature or to about room temperature. Then, the resulting gel is contacted with a solution comprising an anti-cross-linking agent and/or a gellant and/or mixtures thereof. This results in the imbibition, diffusion, and/or adsorption of the surrounding solution into the gel network. Then, the resulting gel is irradiated. The resulting irradiated gel can be heated to a temperature at which it flows, thereby forming an injectable hydrogel formulation, which is suitable for in vivo use. The injectable hydrogel formulations and the solvents, according to the instant invention, are biocompatible and are made suitable for in vivo use.

Alternatively, the polymer solution is gelled by changing the temperature to about 0° C. or to below 0° C. If the hydrogel is formed by heating above the solidification temperature, then changing the temperature will require heating, if the hydrogel is formed by cooling below its solidification temperature, then changing will require cooling. Alternatively, the polymer solution is placed under pressure or in a sensitizing environment, in inert gas or under vacuum with or without changing the chemical environment such as pH, ionic strength and temperature.

According to some aspects and embodiments of the invention, the polymer solution is gelled and reheated above or below the solidification and/or melting temperature sequentially for multiple times.

According to one aspect of the invention, the polymer solution is made with a solvent or a combination of solvents that dissolve the polymer. This polymer solution may contain one or more anti-cross-linking agent. The polymer solution can be gelled by one of the following methods:
by mixing with solution of one or more gellants;
by thermal cycling (cooling and heating or heating and cooling sequentially including the so-called freeze-thaw method);
by irradiation (with or without initiator and/or cross-linking agent and/or anti-cross-linking agent); and/or
by heat treatment (with or without initiator and/or cross-linking agent and/or anti-cross-linking agent).

The resulting gel from any of the above methods can be processed subsequently in the dry, partially dry or fully hydrated state:
by dehydration alone;
by dehydration followed by annealing;
by irradiation;
by application of a magnetic or electric field;
by mechanical deformation; and/or
by high pressure treatment.

These methods for gel formation and post-gel processing can be used alone or in combination in any order. Alternatively, these methods can be used sequentially in any order and/or multiple times. These methods can be followed by partial or complete hydration. Hydration before and/or after gelation and/or post-processing can be in water, aqueous salt solutions such as sodium chloride, potassium chloride, alcohols such as ethanol, methanol, isopropyl alcohol, alcohol solutions, oligomer solution, polyethylene glycol solution or mixtures thereof. These solutions may contain contrast agents (for example, barium salts, iodine, and the like) for x-ray imaging, magnetic resonance imaging, and computed tomography.

The resulting gel and/or post-treated gel is contacted with a solution comprising an anti-cross-linking agent and/or a gellant and/or mixtures thereof. This results in the imbibition, diffusion, and/or adsorption of the surrounding solution into the gel network. Then, the resulting gel is irradiated. The resulting irradiated gel can be brought to a temperature and physical/chemical environment at which it flows, thereby forming an injectable hydrogel formulation, which is suitable for in vivo use. The injectable hydrogel formulations and the solvent therein are biocompatible and are made suitable for in vivo use. Alternatively, the solid irradiated gel comprising one or more anti-cross-linking agents can be used in vivo without melting or liquefication.

According to an aspect of the invention, the hydration solution or the imbibing solution used in the above gels contains anti-cross-linking agent to a concentration of 0.0001 ppm to 1000000 ppm, preferably about 1 to 10000 ppm, or about 100 to 10000 ppm, most preferably about 5000 ppm. The gels can be contacted with the hydration or imbibing solution for 1 second to 1 year, preferably about 1 min to 1 week, most preferably about 10 minutes to 1 week, or about 1 day. Hydration or imbibition can be performed at about −20° C. to about 100° C., or about 0° C. to about 60° C., most preferably about room temperature or body temperature.

According to an aspect of the invention, the solution in which a gel is imbibed before or during irradiation contains polyethylene glycol (PEG) of a single molecular weight or multiple molecular weights. The molecular weight of PEG can vary between 100 g/mol to about 100,000 g/mol, preferably about 200 g/mol to about 1000 g/mol, most preferably about 200 g/mol to 600 g/mol or any integer thereabout or therebetween. The concentration of each molecular weight can vary from 0.0001 w % to about 100 w %, or any fraction thereabout or therebetween.

Physical or chemical cross-linking of a polymer solution or gel can be such that the cross-link degree is low enough that the cross-linked network can still flow when brought to the melting temperature and/or contacted with a solvent or a mixture of solvents.

According to one aspect of the invention, the injectable hydrogel formulations or compositions are prepared with one or more of the above listed solvents, which are biocompatible. According to some aspects and embodiments of the invention, all solvents that are used in the hydrogel, hydrogel formulation or composition are biocompatible solvent in order to form a biocompatible injectable hydrogel formulation or composition, which are suitable for in vivo use.

According to another aspect of the invention, there can be one or more steps in preparing the injectable hydrogel formulations or compositions, which involve exchange of one or more of the above listed solvents, some of which may not be biocompatible, with a biocompatible solvent or a combination of biocompatible solvents. Alternatively, any of the solvents in the hydrogel, hydrogel formulation or composition are exchanged with a biocompatible solvent in order to form an injectable hydrogel formulation or composition, which is suitable for in vivo use.

The term “anti-cross-linking agent” refers to compounds which prevent, inhibit, minimize, attenuate, or reduce the formation of covalent bonds between polymer chains that would otherwise be a result of irradiation, or other agents or procedures for forming cross-links, such as thermal cross-linking, crystallization, and ionic interactions. Polymer chains can be covalently bonded through ionic bonds or the recombination of free radicals induced by heat, radiation or chemical means. An anti-cross-linking agent hinders at least one of these mechanisms. According to the invention, anti-cross-linking agents include compounds with antioxidant and/or free-radical scavenger properties, for example, vitamin C (ascorbic acids) including ester and acetate forms of vitamin C. Anti-cross-linking agent also include compounds with no apparent antioxidant properties, such as organic or inorganic salts, such as calcium chloride, magnesium chloride, phenyl chloride, or hydroxides, peroxides, hydroperoxides, persulfates, and the like.

Antioxidants also include the family of carotenoid compounds, lipoic acid; vitamins such as Vitamins E, D, and B; glutathione; quinones; quinines; amino acids such as arginine, cysteine, tryptophan; peroxides; citric acids; succinic acids; phytochemicals such as ferulic acid, lycopene, lumenene; enzymes such as superoxide dismutase, catalase and glutathione peroxidase; phenolic compounds such as α-tocopherol.

PEG is known as a gellant for vinyl polymers and can inhibit or prevent cross-linking, although it is not known as an anti-cross-linking agent. For example, for 115,000 g/mol PVA of 17.5 wt/v %, 400 g/mol PEG does not inhibit cross-linking at 5 wt % PEG. For PVA having the same molecular weight and concentration, 200 g/mol PEG does not gel the PVA below 25% but inhibits cross-linking when the gel is subjected to 25 kGy of gamma irradiation. PEG can be used in conjunction with anti-cross-linking agents. Accordingly, formulations with PEG and formulations without PEG are aspects of the invention.

Vitamin C (ascorbic acids) is an antioxidant, which also acts as a free radical scavenger. It is hydrophilic, therefore the vitamin C is soluble in aqueous PVA solutions or PVA-based hydrogels.

In one embodiment, the invention relates to an injectable hydrogel formulation wherein the concentration of the anti-cross-linking agent (for example, one that can scavenge free radicals and/or has antioxidant properties) in the polymer solution is enough to facilitate the injectability of the polymer solution after irradiation. For example, the concentration of the anti-cross-linking agent preferably is at least about 1000 ppm or more. The concentration of the anti-cross-linking agent can be above about 0.001 ppm to about 100,000 ppm, preferably between about 1000 ppm and about 10,000 ppm, or any number thereabout or therebetween.

Since PVA is typically dissolved in a hydrophilic solvent, a hydrophobic anti-cross-linking agent such as vitamin E may be solubilized in the polymer solution by using a surfactant. The surfactant can be from the family of Tween surfactants such as Tween 80™ (polyethylene glycol sorbitan monooleate), Tween 20™ (polyethylene glycol sorbitan monolaurate), pluronic® surfactants such as Pluronic F127, poly(ethylene glycol) or any other surfactant that is able to emulsify the hydrophobic or lipophylic anti-cross-linking agent.

According to another aspect of the invention, the irradiation or sterilization is carried out by UV, gamma, e-beam irradiation or by any other source of ionizing radiation.

According to another aspect of the invention, the injectable hydrogel formulations or compositions can be sterilized by methods other than radiation sterilization such as ethylene oxide gas, gas plasma or autoclave sterilization or by sterile filtration and the like.

According to one aspect of the invention, the radiation dose is at least about 1 kGy, for example, about 25 kGy, between 25 and 1000 kGy, about 50 kGy, about 100 kGy, and about 150 kGy. According to another aspect of the invention, the radiation dose rate is about 0.001 kGy/min to 10000 kGy/min, preferably 0.1 kGy/min to 100 kGy/min, most preferably from about 1 kGy/min to 25 kGy/min, or about 12 kGy/min. According to another aspect of the invention, the radiation temperature is from about −196° C. to about 500° C., preferably from about −20° C. to about 100° C., most preferably from about −20° C. to about 50° C., or about room temperature. According to another aspect of the invention, the radiation dose can be applied in a single application or in multiple applications (for example, sequential).

The injectable hydrogel formulation can have various viscosities. The viscosity of an injectable hydrogel formulation can be low enough to pass through an injection needle. Size of the needle can vary, for example, a needle 10 size of about 33, about 28, about 25, about 22, about 20, about 18 or about 14 gauge or lower, or any size thereabout or therebetween. The inner diameter of the needle also can vary, for example, an inner diameter of about 0.025 mm or more, about 0.089 mm or about 0.10 mm or more, or any diameter thereabout or therebetween.

Injectable hydrogel formulations include monomer, polymer, polymer blends, or copolymers of polyvinyl alcohol (PVA), polyvinyl pyrrolidone (PVP), polyacrylamide (PAAm), polyacrylic acid (PAA), alginates, polysaccharides, polyoxyethylene-polyoxypropylene co-polymers, poly-N-alkylacrylamides, poly-N-isopropyl acrylamide (PNIPAAm), poly(ethylene glycol) methacrylate, poly(ethylene-co-vinyl alcohol) or a polyolefin such as polyethylene.

Injectable hydrogel formulations also include hydrogels made of a vinyl polymer, such as poly(vinyl alcohol), poly(vinyl pyrrolidone), an acrylamide polymer such as poly(N-isopropyl acrylamide), an acrylic polymer such as poly(acrylic acid), poly(ethylene glycol) methacrylate, poly(ethylene-co-vinyl alcohol), a polyolefin such as polyethylene, wherein one of the polymers is grafted on another one.

The term “cross-link-resistant” as defined herein, in the context of a cross-link-resistant injectable hydrogel formulation, refers to a degree of resistance of the injectable hydrogel formulation to cross-linking when the hydrogel is the subject of irradiation or other agents or procedures that can cause cross-linking. The resistance to cross-linking facilitates injectability of the hydrogel formulation, wherein the anti-cross-linking agent is present, for example, during irradiation, to partially or practically wholly prevent, inhibit, minimize, attenuate, or reduce cross-linking of the hydrogel formulation, thereby rendering the hydrogel formulation injectable.

In some embodiments, injectable hydrogel formulation is prepared by starting with an aqueous PVA solution (at least about 1 wt % PVA, above about 1 wt % PVA, about 5 wt % PVA, about 10 wt % PVA, above about 10 wt % PVA, about 15 wt % PVA, about 20 wt % PVA, about 25 wt % PVA, about above 25 wt % PVA) and mixing it with an anti-cross-linking agent at an elevated temperature (for example, above about 50° C.). Upon cooling down to below the solidifying temperature or to about room temperature, the mixture will form a solid hydrogel formulation. This solid hydrogel formulation can be irradiated. The hydrogel formulation is injectable when it is above the melting temperature of the hydrogel, for example from 40 to 120° C., or 50 or 70° C. For example, a PVA-based hydrogel comprising a solvent, an anti-cross-linking agent and optionally PEG. This hydrogel is heated to above about 40 to 120° C. and subsequently cooled down to a temperature above about −196° C., above about −20° C., above about 0° C., preferably about room temperature or body temperature for about 5 minutes or more. Temperatures close to body temperature are preferred for use in in situ injection.

In some aspects and embodiments of the invention where gel formation and/or post-processing methods are used, the resulting hydrogel formulation is injectable when it is above or below solidification temperature of the hydrogel (depending on whether the formulation is in liquid form above or below the solidification temperature), for example from 40 to 120° C., or 50 or 70° C. For example, a PVA-based hydrogel comprising a solvent, an anti-cross-linking agent and optionally PEG. This hydrogel is heated to above melting temperature of the hydrogel, for example, above about 40 to 120° C. and subsequently cooled down to a temperature above about −196° C., above about −20° C., above about 0° C., preferably about room temperature or body temperature for about 5 minutes or more. Temperatures close to body temperature are preferred for use in in situ injection.

The ingredients of a hydrogel formulation, irradiation of the hydrogel formulation, irradiation dose, dose rate, irradiation temperature, pressure during gelation and pressure during melting, melting environment, such as vacuum, gas or liquid, can change melting temperature and/or solidification temperature. The initial temperature at which a polymer solution is made also can change the subsequent solidification and/or melting temperatures of the same formulation.

It is desirable that a hydrogel formulation is, or becomes and remains solid at body temperature and/or environment inside the bodily cavity, into which injection or implantation of the hydrogel formulation is done. In order to obtain fast gelation and to prevent damage to bodily tissues, it is desirable that injection temperature is close to body temperature, for example within 2 to 33° C., preferably about 10° C. For example, one hydrogel formulation can be injected at 45° C., after injection, upon cooling down to body temperature in the body environment, this formulation will become a solid gel. Such a hydrogel formulation exhibits upper critical solution temperature behavior. That is, above certain temperature, the components are miscible and form a continuous, flowing phase. Another hydrogel formulation can be injected at 30° C., after injection, upon heating up to body temperature in the body environment, this formulation will become a solid gel. Such hydrogel formulation exhibits lower critical solution temperature behavior. That is, below certain temperature, the components are miscible and form a continuous and a flowing phase.

In some embodiments poly(vinyl alcohol) (PVA) can be used as the base hydrogel. The base PVA hydrogel can be prepared by a freeze-thaw method by subjecting a PVA solution (PVA can be dissolved in solvents such as water or DMSO) to one or multiple cycles of freeze-thaw. PVA solution used in the freeze-thaw method can contain another ingredient like an anti-cross-linking agent and optionally PEG. The base PVA hydrogel can also be prepared by radiation cross-linking of a PVA solution.

According to an aspect of the invention, the molecular weight of PVA can be between 2,000 to 400,000 g/mol, preferably between 16,000 and 250,000 g/mol, or any number thereabout or therebetween.

According to another aspect of the invention, the molecular weight of PEG can be between 100 to 10,000 g/mol, preferably 200 to 6000 g/mol, or any number thereabout or therebetween.

According to an aspect, polyvinyl alcohol aqueous solution is prepared with PEG at an elevated temperature. The mixture is placed in a gamma sterilizable container and cooled down to room temperature. Upon cooling down, the PVA-based hydrogel is formed with the PEG and possibly some excess liquid composed of solvent and PEG. This mixture also is prepared with vitamin C in either the PVA solution or the PEG, so that there is vitamin C in the final hydrogel formulation. The container that contains the PVA gel with the PEG and some excess liquid along with vitamin C is sealed and gamma sterilized. In the operating room, the container, such as syringe containing the injectable hydrogel formulation, is heated to above the gel solution temperature (for example, above 70° C., preferably about 90 to about 95° C.). At this elevated temperature the hydrogel is softened or dissolved, and later is injected into a cavity in the human or animal body. The PVA-based hydrogel formulation contains vitamin C as an anti-oxidant and PEG as a gellant; therefore re-gelation can take place inside this cavity. This aspect shows how a hydrogel or a hydrogel formulation can be prepared with an antioxidant such as vitamin C so that it can be gamma sterilized, without compromising the injectability of the hydrogel or the hydrogel formulation, thereby preventing, inhibiting, minimizing, attenuating, or reducing the cross-linking of the hydrogel during the sterilization, so that the hydrogel or the hydrogel formulation can be melted later during surgery and injected into a body cavity. The anti cross-linking agent can be added also to decrease the viscosity for ease of injection. The viscosity in its absence would be higher.

In some of the embodiments poly-N-isopropyl acrylamide (PNIPAAm) also can be used as the base hydrogel. The base PNIPAAm hydrogel can be prepared by radiation cross-linking of a PNIPAAm solution. Alternatively, the methods described by Lowman et al. can be used.

According to an aspect, a copolymer of PNIPAAm with monomers/polymers such as acrylic acid, hydroxyethyl methacrylate, PVA, or PVP aqueous solution is prepared at room temperature. The mixture is placed in a gamma sterilizable container. This mixture also is prepared with vitamin C. The container that contains the PNIPAAm solutions with vitamin C is sealed and gamma sterilized. PNIPAAm solutions have a lower critical solution temperature (LCST), which may be at around body temperature depending on the copolymer or blend composition. At and above this temperature, they physically associate and form a gel. In the operation room, the sterilized container, such as syringe containing the injectable hydrogel formulation, is injected into a cavity in the human or animal body at below this LCST. The solution contains hydrogel, vitamin C as an anti-cross-linking agent therefore gelation can take place inside this cavity. This aspect shows how a hydrogel or a hydrogel formulation showing critical solubility behavior can be prepared with an anti-cross-linking agent such as vitamin C so that it can be gamma sterilized, without compromising the injectability of the hydrogel or the hydrogel formulation, thereby preventing, inhibiting, minimizing, attenuating, or reducing the cross-linking of the hydrogel during the sterilization, so that the hydrogel or the hydrogel formulation can be injected later during surgery into a body cavity.

In some of the embodiments a topological gel (TP) can be used as the base hydrogel. The base TP hydrogel can be prepared by methods described by Tanaka et al. (Progress in Polymer Science, 2005, 30, 1-9). The polymer chains in TP gels are flexibly bound by cross-linkers that are sliding along the individual chain.

### Definitions and Other Embodiments:

The terms “about” or “approximately” in the context of numerical values and ranges refers to values or ranges that approximate or are close to the recited values or ranges such that the invention can perform as intended, such as having a desired degree of cross-linking, as is apparent to the skilled person from the teachings contained herein. This is due, at least in part, to the varying properties of polymer compositions. Thus, these terms encompass values beyond those resulting from systematic error. These terms make explicit what is implicit.

The term “contact” refers to physical proximity with or touching, mixing, blending, doping, diffusing, imbibing, and/or soaking of one ingredient with another. For example, a PVA hydrogel in contacted with an anti-cross-linking agent, or a PVA hydrogel is diffused, adsorbed, imbibed, and/or soaked with a solution of an anti-cross-linking agent or a mixture of anti-cross-linking agents.

Contacting also refers to placing the hydrogel sample in a specific environment for a sufficient period of time at an appropriate temperature, for example, contacting the hydrogel sample with a solution of an anti-cross-linking agent or a mixture of anti-cross-linking agents. The environment is heated to a temperature ranging from room temperature to a temperature below the melting point of the hydrogel material. The contact period ranges from at least about 1 minute to several weeks and the duration depending on the temperature of the environment.

The term “Mechanical deformation” refers to a deformation taking place on the solid form of the material, essentially ‘cold-working’ the material. The deformation modes include uniaxial, channel flow, uniaxial compression, biaxial compression, oscillatory compression, tension, uniaxial tension, biaxial tension, ultra-sonic oscillation, bending, plane stress compression (channel die), torsion or a combination of any of the above. The deformation could be static or dynamic. The dynamic deformation can be a combination of the deformation modes in small or large amplitude oscillatory fashion. Ultrasonic frequencies can be used. All deformations can be performed in the presence of sensitizing gases and/or at elevated temperatures.

The term “hydrogel”, as described herein, encompasses polymer-based hydrogels, including PVA-based hydrogels and all other hydrogel formulations disclosed herein including de-hydrated hydrogels. PVA-hydrogels are networks of hydrophilic polymers containing absorbed water that can absorb a large amounts of energy, such as mechanical energy, before failure.

The term “injectable hydrogel formulation” refers to a hydrogel formulation or composition having a viscosity such that can pass through an injection needle, as described herein. A hydrogel formulation can comprise polymeric and non-polymeric components and one or more solvents, which under certain conditions can form a hydrogel. These conditions can be defined by factors such as the ingredients of the formulation, temperature, pressure, pH, ionic strength, environment such as vacuum, gas and/or liquid, electromagnetic environment and/or irradiation. A hydrogel formulation also used in reference to a solid or liquid form of a hydrogel.

The term “injectable hydrogel” has been used as shorthand term in the field to refer to hydrogel solutions or compositions, which are capable of forming hydrogels under suitable condition. The “injectable hydrogel”, in fact, is a pre-gel formulation, which can undergo physicochemical and/or structural changes under suitable conditions and become a hydrogel. The pre-gel also can be a loosely associated ‘hydrogel-like’ form. For example, an injectable hydrogel formulation, which has been called as “injectable hydrogel”, can be flowable under gravity, flowable under additional forces such as an applied pressure, or can be a fluid-like, injectable, biocompatible pre-gel material (having all the ingredients to form a hydrogel and a viscosity such that can pass through an injection needle), that becomes a hydrogel upon injection as a result of physicochemical and/or structural changes under suitable condition, such as in vivo in human or animal body temperature.

A hydrogel under certain environmental conditions can be transformed into liquid phase, in which it flows and is injectable (solution, formulation and the like). Such conditions can be defined by environmental factors such as the ingredients of the formulation, temperature, pressure, pH, ionic strength, environment such as vacuum, gas and/or liquid, electromagnetic environment and/or irradiation.

The term “hydrogel solution” also refers to a solution comprising a monomer, polymer, mixture of monomer and/or polymers, co-polymers, networks of hydrophilic polymers, a polymer formulation containing other ingredients, that is in a non-solid, injectable, liquid or flowable form, flowable under a force such as pressure, and capable of forming hydrogel under suitable conditions. A hydrogel solution can be a hydrogel formulation in applicable circumstance.

The term “heating” refers to thermal treatment of the polymer at or to a desired heating temperature. In one aspect, heating can be carried out at a rate of about 10° C. per minute to the desired heating temperature. In another aspect, the heating can be carried out at the desired heating temperature for desired period of time. In other words, heated polymers can be continued to heat at the desired temperature, below or above the melt, for a desired period of time. Heating time at or to a desired heating temperature can be at least 1 minute to 48 hours to several weeks long. In one aspect the heating time is about 1 hour to about 24 hours. In another aspect, the heating can be carried out for any time period as set forth herein, before or after irradiation. Heating temperature refers to the thermal condition for heating in accordance with the invention. Heating can be performed at any time in a process, including during, before and/or after irradiation.

The term “annealing” refers to heating or a thermal treatment condition of the polymers in accordance with the invention. Annealing generally refers to continued heating the polymers at a desired temperature below its peak melting point for a desired period of time. Annealing time can be at least 1 minute to several weeks long. In one aspect the annealing time is about 4 hours to about 48 hours, preferably 24 to 48 hours and more preferably about 24 hours. “Annealing temperature” refers to the thermal condition for annealing in accordance with the invention. Annealing can be performed at any time in a process, including during, before and/or after irradiation.

In certain embodiments of the present invention in which annealing can be carried out, for example, in an inert gas, e.g., nitrogen, argon or helium, in a vacuum, in air, and/or in a sensitizing atmosphere, for example, acetylene.

“Melting temperature of a hydrogel” refers to a temperature at which a transformation occurs in a hydrogel from solid to liquid-like state. In the liquid-like state, the interactions between polymer chains in the hydrogel formulation are not as strong as in the solid state and this will manifest itself in physical terms as softening and eventually flow. Melting temperature can be from about −20° C. to about 200° C., or from about 0° C. to about 130° C., or from about 10° C. to about 100° C.

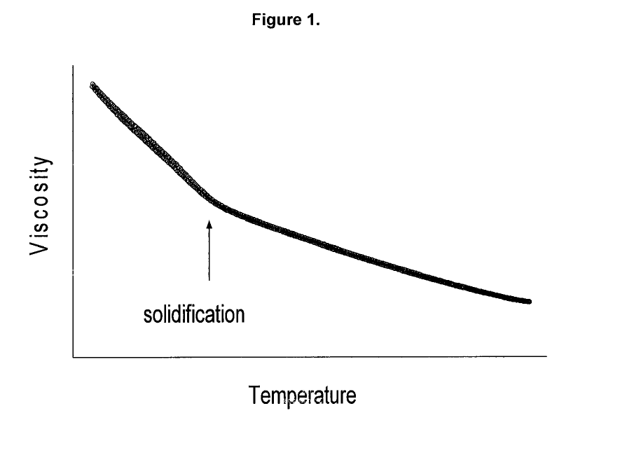

The term “solidifying temperature” generally refers to a temperature above or below which the mobility of the polymer chains is restricted such that the polymer solution becomes mostly solid and non-flowing. “Solidification temperature of a hydrogel” refers to the temperature at which a transformation occurs in a hydrogel from liquid-like to solid state. In the solid state, the interactions between polymer chains in the hydrogel formulation are stronger than in the liquid-like state and this will manifest itself in physical terms as the inability to flow in one-phase. At this temperature, there is an observable change in the rate of viscosity change as a function of temperature (see for example, FIG. 1). Solidification temperature can be from about −20° C. to about 200° C., or from about 0° C. to about 130° C., or from about 10° C. to about 100° C. Solidification and melting temperature of a hydrogel or hydrogel formulation are not necessarily the same.

In one aspect of the invention, the type of “radiation”, preferably ionizing, is used. According to another aspect of the invention, a dose of ionizing radiation ranging from about 25 kGy to about 1000 kGy is used. The radiation dose can be about 25 kGy, about 50 kGy, about 65 kGy, about 75 kGy, about 100 kGy, about 150, kGy, about 200 kGy, about 300 kGy, about 400 kGy, about 500 kGy, about 600 kGy, about 700 kGy, about 800 kGy, about 900 kGy, or about 1000 kGy, or above 1000 kGy, or any value thereabout or therebetween. Preferably, the radiation dose can be between about 25 kGy and about 150 kGy or between about 50 kGy and about 100 kGy. These types of radiation, including gamma and/or electron beam, kills or inactivates bacteria, viruses, or other microbial agents potentially contaminating medical implants, including the interfaces, thereby achieving product sterility. The irradiation, which may be electron or gamma irradiation, in accordance with the present invention can be carried out in air atmosphere containing oxygen, wherein the oxygen concentration in the atmosphere is at least 1%, 2%, 4%, or up to about 22%, or any value thereabout or therebetween. In another aspect, the irradiation can be carried out in an inert atmosphere, wherein the atmosphere contains gas selected from the group consisting of nitrogen, argon, helium, neon, and the like, or a combination thereof. The irradiation also can be carried out in a sensitizing gas such as acetylene or mixture or a sensitizing gas with an inert gas or inert gases. The irradiation also can be carried out in a vacuum. The irradiation can also be carried out at room temperature, or at between room temperature and the melting point of the polymeric material, or at above the melting point of the polymeric material. Subsequent to the irradiation step the hydrogel can be melted or heated to a temperature below its melting point for annealing. These post-irradiation thermal treatments can be carried out in air, PEG, solvents, non-solvents, inert gas and/or in vacuum. Also the irradiation can be carried out in small increments of radiation dose and in some embodiments these sequences of incremental irradiation can be interrupted with a thermal treatment. The sequential irradiation can be carried out with about 1, 10, 20, 30, 40, 50, 100 kGy, or higher radiation dose increments. Between each or some of the increments the hydrogel can be thermally treated by melting and/or annealing steps. The thermal treatment after irradiation may eliminate the residual free radicals in the hydrogels created by irradiation, and/or eliminate the crystalline matter, and/or help in the removal of any extractables that may be present in the hydrogel.

According to another aspect of this invention, the irradiation may be carried out in a sensitizing atmosphere. This may comprise a gaseous substance which is of sufficiently small molecular size to diffuse into the polymer and which, on irradiation, acts as a polyfunctional grafting moiety. Examples include substituted or unsubstituted polyunsaturated hydrocarbons; for example, acetylenic hydrocarbons such as acetylene; conjugated or unconjugated olefinic hydrocarbons such as butadiene and (meth)acrylate monomers; sulphur monochloride, with chloro-tri-fluoroethylene (CTFE) or acetylene being particularly preferred. By “gaseous” is meant herein that the sensitizing atmosphere is in the gas phase, either above or below its critical temperature, at the irradiation temperature.

At any step of the manufacturing, the hydrogel can be irradiated by e-beam or gamma to cross-link. The irradiation can be carried out in air, in inert gas, in sensitizing gas, or in a fluid medium such as water, saline solution, polyethylene-glycol solution, and the like. The radiation dose level is between one kGy and 10,000 kGy, preferably 25 kGy, 40 kGy, 50 kGy, 200 kGy, 250 kGy, or above.

The term “dose rate” refers to a rate at which the radiation is carried out. Dose rate can be controlled in a number of ways. One way is by changing the power of the e-beam, scan width, conveyor speed, and/or the distance between the sample and the scan horn. Another way is by carrying out the irradiation in multiple passes with, if desired, cooling or heating steps in-between. With gamma and x-ray radiations the dose rate is controlled by how close the sample is to the radiation source, how intense is the source, the speed at which the sample passes by the source.

The dose rate of the electron beam can be adjusted by varying the irradiation parameters, such as conveyor speed, scan width, and/or beam power. With the appropriate parameters, a 20 Mrad melt-irradiation can be completed in for instance less than 10 minutes. The penetration of the electron beam depends on the beam energy measured by million electron-volts (MeV). Most polymers exhibit a density of about 1 g/cm³, which leads to the penetration of about 1 cm with a beam energy of 2-3 MeV and about 4 cm with a beam energy of 10 MeV. The penetration of e-beam is known to increase slightly with increased irradiation temperatures. If electron irradiation is preferred, the desired depth of penetration can be adjusted based on the beam energy. Accordingly, gamma irradiation or electron irradiation may be used based upon the depth of penetration preferred, time limitations and tolerable oxidation levels.

Ranges of acceptable dose rates are exemplified in International Application WO 97/29793. In general, the dose rates vary between 0.005 Mrad/pass and 50 Mrad/pass. The upper limit of the dose rate depends on the resistance of the polymer to cavitation/cracking induced by the irradiation.

If electron radiation is utilized, the energy of the electrons also is a parameter that can be varied to tailor the properties of the irradiated polymer. In particular, differing electron energies result in different depths of penetration of the electrons into the polymer. The practical electron energies range from about 0.1 MeV to 16 MeV giving approximate iso-dose penetration levels of 0.5 mm to 8 cm, respectively. The preferred electron energy for maximum penetration is about 10 MeV, which is commercially available through vendors such as Studer (Daniken, Switzerland) or E-Beam Services New Jersey, USA). The lower electron energies may be preferred for embodiments where a surface layer of the polymer is preferentially cross-linked with gradient in cross-link density as a function of distance away from the surface.

“Sterilization”, one aspect of the present invention discloses a process of sterilization of cross-link resistant hydrogels, such as irradiation cross-link resistant injectable PVA-hydrogel formulations. The process comprises sterilizing the hydrogels by ionizing sterilization with gamma or electron beam radiation, for example, at a dose level ranging from about 25-70 kGy, or by gas sterilization with ethylene oxide or gas plasma.

Another aspect of the present invention discloses a process of sterilization of irradiation cross-link resistant injectable hydrogel formulations, such as injectable PVA-hydrogel formulation. The process comprises sterilizing the injectable hydrogel formulations by ionizing sterilization with gamma or electron beam radiation, for example, at a dose level ranging from 25-200 kGy.

The invention is further described by the following examples, which do not limit the invention in any manner.

### EXAMPLES

### Example 1

### Preparation and Irradiation of a PVA Solution by Ionizing Radiation

A 17.5 wt/v % of polyvinyl alcohol (PVA, Molecular weight=115,000 g/mol, Scientific Polymer Products, Ontario, N.Y.) was prepared by dissolving PVA in deionized water at 90° C. by constant stirring. The solution was kept at 90° C. in an air convection oven for 6 hours for degassing.

At this molecular weight of PVA and at this PVA concentration, the solution was very viscous at 90° C.

For sterilization, the solution that was kept in the oven was poured into 10 cc disposable syringes (Terumo Corp, Tokyo, Japan) that were pre-heated to 90° C. They were covered with Parafilm® and packaged in vacuum (Rival Products, VS110-BCD, El Paso, Tex.). These syringes were gamma irradiated to 25 kGy and 100 kGy (Steris, Northborough, Mass.). Controls were unirradiated.

### Example 2

### Measurement of Viscosity by Using Bubble Tubes

The viscosity of unirradiated and irradiated PVA solutions were determined by using bubble tubes (Fisher Scientific). This method was appropriate because of the very high viscosity of the solutions. The bubble tubes were calibrated with viscosity standards (N100, D5000, S8000, N15000, Koehler Instrument Company, Bohemia, N.Y.).

Liquid samples were poured into the bubble tubes slowly without the formation of bubbles until the fill line. The cork cap was tightly fitted and the entire tube was vacuum packaged in a plastic pouch to prevent the sample from leaking. Then the samples were placed in a water bath at 50° C. or 100° C. The tubes were inverted and reverted. The time that it took the bubble volume between the two designated lines to travel 10 cm was recorded (between the bottom and first top lines). At least 6 measurements were done for each sample by two different observers.

### Example 3

### Viscosity of Unirradiated PVA Solutions and Gel Content of Irradiated PVA Solutions

PVA solutions were prepared at a concentration of 17.5 wt/v % in deionized water as described in Example 1. Four different molecular weights of PVA were used: 16,000; 61,000; 86,000; and 115,000 g/mol. These solutions were poured into pre-heated syringes at 90° C. and packaged in vacuum. The syringes were then gamma irradiated to 25 kGy.

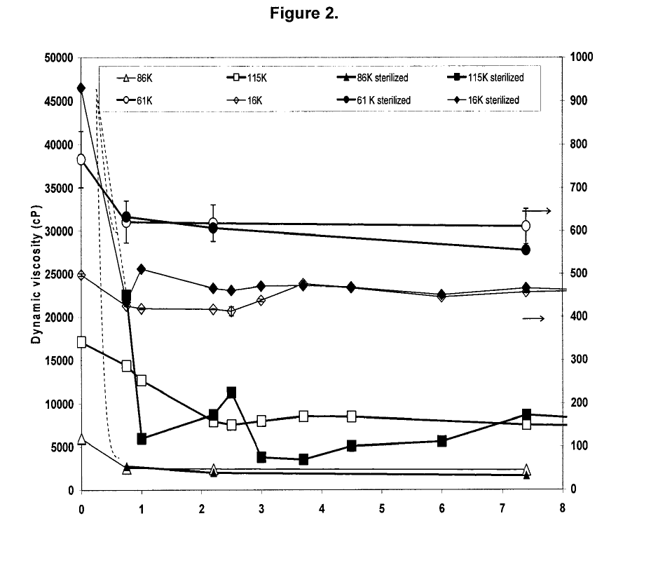

Pure PVA solutions were viscous but free flowing liquids at 50° C. The viscosities, as measured by using bubble tubes, were 498±3, 766±5, 5976±65, 17144±715 centiPoise (cP) for PVA molecular weights of 16K,000; 61,000; 86,000 and 115,000 respectively (see FIG. 2).

When these PVA solutions were irradiated to 25 kGy, only the solution containing PVA of molecular weight 16,000 g/mol was a liquid at 50° C. The viscosity of this solution was 931±45 cP. The sterilized PVA solutions containing higher molecular weight PVA than 16,000 g/mol did not flow at temperatures up to 120° C., indicating that these solutions were cross-linked by the gamma radiation.

While physically cross-linked or entangled networks of unirradiated PVA became liquid at temperatures ranging from room temperature to 95° C. depending on molecular weight and concentration, irradiated and chemically cross-linked gels did not dissolve and flow at temperatures up to 120° C. For these samples, the gel content was calculated in the following manner:

The samples were boiled in water for 6 hours. They were taken out of boiling water and weighed hourly to ensure equilibrium swelling in boiling water. The samples were then placed in an air convection oven at 90° C. for at least 22 hours. The final dry weight was recorded. The gel content was the ratio of dry weight to swollen weight.

The gel contents of sterilized PVA gels containing PVA with molecular weight of 61,000, 86,000, and 115,000 g/mol were 12.0±0.4%, 13.8±0.8%, and 14.9±4.9% respectively. These results showed that the solutions of PVA with varying molecular weights were all chemically cross-linked during irradiation.

### Example 4

### Viscosity of Unirradiated and Sterilized (25 kGy) PVA Solutions Containing Vitamin C

PVA solutions at a concentration of 17.5 wt/v % were prepared as described in Example 1. Four different molecular weights of PVA were used: 16,000; 61,000; 86,000 and 115,000 g/mol. Vitamin C powder (L-ascorbic acid, 99.2%, Fisher Scientific, Houston, Tex.) was mixed into the PVA solutions at a Vitamin C to PVA repeating unit ratio of 0.75, 1.0, 2.2, 2.5, 3.0, 3.7, 4.5, 6.0, 7.4, and 10.4 mol/mol for PVA solutions of molecular weight 16,000 and 115,000 and at ratios of 0.75, 2.2, and 7.4 mol/mol for PVA solutions of molecular weight 61,000 and 86,000.

These solutions were poured into pre-heated syringes at 90° C. and packaged in vacuum. The syringes were then gamma irradiated to 25 kGy.

In contrast to control PVA sterilized solutions containing PVA of molecular weight 61,000, 86,000 and 115,000 g/mol, which were chemically cross-linked into a gel network, vitamin C containing sterilized PVA solutions were not cross-linked into gel networks and flowed at 50° C. (FIG. 2). The viscosity of the sterilized PVA solution containing PVA of molecular weight 16K showed significant increase compared to unirradiated solution, suggesting a certain degree of cross-linking. When this solution contained vitamin C, this increase was not observed, indicating the anti-cross-linking effect of vitamin C. At higher molecular weights, the PVA solutions without vitamin C did not flow after irradiation at temperatures up to 120° C. In contrast, when vitamin C was added all of these PVA solutions with higher molecular weights showed negligible changes in viscosity, indicating the anti-cross-linking effect of vitamin C.

Anti-cross-linking effect of vitamin C on the viscosity of sterilized PVA solutions containing 17.5 wt/v % PVA with molecular weights of 16K, 61K, 86K, and 115K is shown in FIG. 2.

### Example 5

### Viscosity of Unirradiated and Irradiated (100 kGy) PVA Solutions Containing Vitamin C

PVA solutions at a concentration of 17.5 wt/v % were prepared as described in Example 1. Two different molecular weights of PVA were used: 16,000; and 115,000 g/mol. Vitamin C powder (L-ascorbic acid, 99.2%, Fisher Scientific, Houston, Tex.) was mixed into the PVA solutions at a Vitamin C to PVA repeating unit ratio of 0.75, 1.0, 2.2, 2.5, 3.0, 3.7, 4.5, 6.0, 7.4, and 10.4 mol/mol.

These solutions were poured into pre-heated syringes at 90° C. and packaged in vacuum. The syringes were then gamma irradiated to 100 kGy.

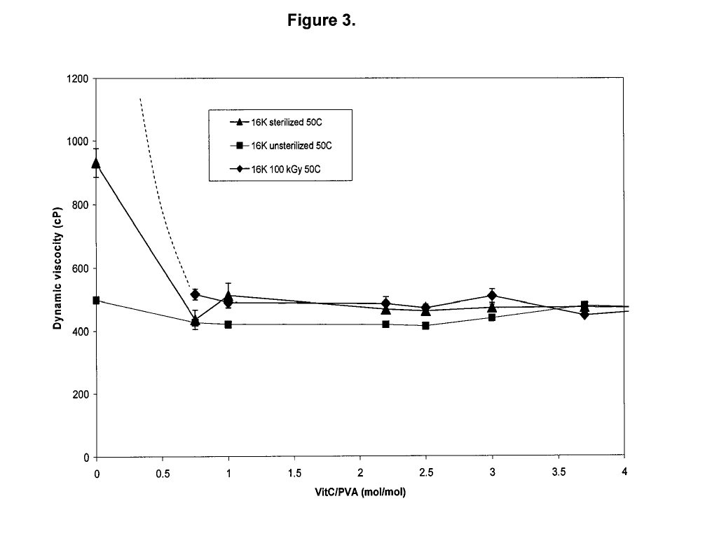

The control PVA solution containing PVA of molecular weight 16,000 g/mol became a chemically cross-linked solid network when irradiated to 100 kGy (see FIG. 3). The gel content of this sample was 13.9±0.5%. This showed that the extent of cross-linking in this solution was higher at 100-kGy irradiation then at 25-kGy irradiation, where the sample was still able to flow. The vitamin C containing solutions, without or with irradiation, were in liquid forms with similar viscosities. This indicates that even the lowest vitamin C concentration was enough to prevent or inhibit the cross-linking of PVA having molecular weight of 16,000 g/mol at a radiation dose of 100 kGy (see FIG. 3).

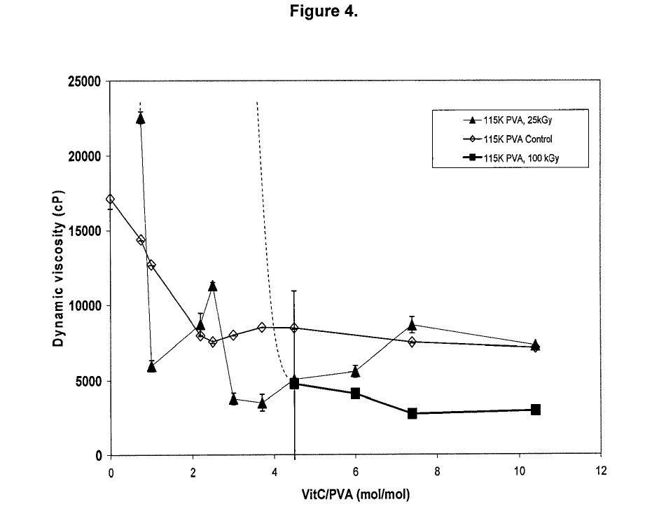

When irradiated to 100 kGy, PVA solutions containing PVA of molecular weight 115,000 g/mol were chemically cross-linked into a gel network with Vitamin C concentrations below a Vitamin C to PVA repeating unit ratio of 4.5 (see FIG. 4). This suggested that vitamin C concentrations below this value were not enough to inhibit cross-linking to a level to enable flow in PVA solutions of molecular weight 115,000 g/mol at this concentration. The irradiated solutions containing vitamin C larger than this value had similar viscosity to unsterilized and gamma-sterilized samples, suggesting minimal or no cross-linking.

The effect of vitamin C on the viscosity of unirradiated, 25 and 100 kGy irradiated PVA solutions containing PVA molecular weight of 16K g/mol is shown in FIG. 2 and FIG. 3.

### Example 6

### Viscosity of Unirradiated and Irradiated (25 kGy) PVA Solutions Containing Polyethylene Glycol

PVA solutions at a concentration of 17.5 wt/v % were prepared as described in Example 1. The molecular weight of PVA was 115,000 g/mol. Polyethylene glycol (Molecular weight 400 g/mol) was mixed into the PVA solutions at a PEG repeating unit to PVA repeating unit ratio of 17, 86, 290, and 639 mol/mol.

All unsterilized PVA-PEG solutions flowed at 100° C. Of the irradiated PVA solutions, only those equal to or above a PEG concentration of PEG to PVA ratio of 290 flowed, suggesting that at PEG concentrations below this value, chemical cross-linking into a gel network was not hindered. The gel content of 25 kGy irradiated PVA-PEG solutions containing a PEG to PVA ratio of 17 and 86 were 2.5±0.9 and 13.9±1.2%, confirming this observation. This result showed that PEG can inhibit or prevent cross-linking of PVA solutions with molecular weight of 115,000 g/mol at certain concentrations.

### Example 7

### Gel Content of Dilute and Concentrated PVA Solutions

PVA solutions at a concentration of 1 and 17.5 wt/v % were prepared as described in Example 1. These solutions were poured into pre-heated syringes at 90° C. and packaged in vacuum. The syringes were then gamma irradiated to 25 kGy and 100 kGy.

The viscosity of unirradiated PVA solutions are shown in Table 1. The gel content of irradiated PVA solutions are shown in Table 2.

TABLE 1
The viscosity of PVA solutions containing 16K and 115K g/mol
and 1 and 17.5 wt/v % PVA at 50° C.

| | 16,000 g/mol | 115,000 g/mol |
|---|---|---|
| 1 wt/v % | 436 ± 1 cP | 406 ± 0 cP |
| 17.5 wt/v % | 498 ± 3 cP | 17144 ± 715 cP |

TABLE 2
The gel content of PVA gels containing 16K and 115K g/mol and
1 and 17.5 wt/v % PVA irradiated to 25 and 100 kGy.

| | 25 kGy | | 100 kGy | |
|---|---|---|---|---|
| | 16,000 | 115,000 | 16,000 | 115,000 |
| | g/mol | g/mol | g/mol | g/mol |
| 1 wt/v % | 1.0 ± 0.4% | 2.8 ± 0.5% | 2.3 ± 0.2% | 6.2 ± 0.4% |
| 17.5 wt/v % | NA | 14.9 ± 4.9% | 13.9 ± 0.5% | 16.7 ± 1.4% |

The results showed that diluting the PVA solution decreased gel content but did not prevent or inhibit cross-linking for 16,000 and 115,000 g/mol PVA solutions (Table 1 and Table 2). Increasing molecular weight resulted in increased cross-link density as indicated by the increase in the gel content at each dose and concentration (Table 2).

### Example 8

### Facilitation of Injectability of a PVA-PEG Gel After Irradiation by Adding Vitamin C

PVA solutions at a concentration of 17.5 wt/v % were prepared as described in Example 1. The molecular weight of PVA was 115,000 g/mol. Polyethylene glycol (Molecular weight 400 g/mol) was mixed into the PVA solutions at a PEG repeating unit to PVA repeating unit ratio of 17 and 86. Vitamin C was added to these solutions at a ratio of vitamin C to PVA repeating unit of 0.75 mol/mol (8800 ppm). The control solution did not contain vitamin C. Then all solutions were further gamma sterilized at 25 kGy.

All unsterilized PVA-PEG solutions flowed at 50° C. The gel content of 25 kGy irradiated control PVA-PEG solutions containing a PEG to PVA ratio of 17 and 86 were 2.5±0.9 and 13.9±1.2%. Vitamin C containing irradiated solution containing the same amount of PVA and PEG flowed at 50° C. and the viscosity was 21132±186 cP and 12163±560 cP. These results showed that PVA solutions containing PEG, which were not injectable after gamma irradiation could be made injectable by the addition of vitamin C before irradiation.

### Example 9

### The Effect of Vitamin E on the Cross-Linking of PVA

PVA solutions at a concentration of 17.5 wt/v % were prepared as described in Example 1. The molecular weight of PVA was 115,000 g/mol. Vitamin E (D,L-α-tocopherol, 98%, DSM Nutritional Products, Poughkeepsie, N.J.) was added to these solutions in the amount of 7500 ppm. It was observed that some of the vitamin E residue settled at the top of the solution, suggesting that not all of this vitamin E was soluble in the polymer solution. Control solution did not contain vitamin E. Then all solutions were further gamma sterilized at 25 kGy.

Neither the control nor the vitamin E-containing irradiated polymer solutions melted at 120° C. This result showed that vitamin E by itself did not inhibit cross-linking in PVA of this molecular weight at this concentration.

### Example 10

### Injectable Formulations with More Than One Molecular Weight of PEG

A 17.5 wt/v % of polyvinyl alcohol (PVA, Molecular weight=115,000 g/mol, Scientific Polymer Products, Ontario, N.Y.) was prepared by dissolving PVA in deionized water at 90° C. by constant stirring. The solution was kept at 90° C. in an air convection oven for 6 hours for degassing. At this molecular weight of PVA and at this PVA concentration, the solution was very viscous at 90° C.

Poly(ethylene glycol) with molecular weight 400 g/mol (PEG400) heated to 90° C. was mixed vigorously with poly(ethylene glycol) of 200 g/mol molecular weight (PEG200) also previously heated to 90° C. The resulting PEG mixture was maintained at about 90° C. for 20 minutes. Then the PEG mixture was mixed further into the PVA solution at 90° C. Mixtures that contained 17.5 w/v % PVA, and 17.5 w/v % PEG400 and 17.5 w/v % PEG200; 39 w/v % PEG400 and 10 w/v % PEG200; 39 w/v % PEG400 and 17.5 w/v % PEG200; 39 w/v % PEG400 and 39 w/v % PEG200 were prepared.

Poly(ethylene glycol) with molecular weight 600 g/mol (PEG600) was first dissolved in water as a 95 w/w % solution, this solution was heated to 90° C. Then the PEG600 solution was mixed vigorously with poly(ethylene glycol) of 200 g/mol molecular weight (PEG200) also previously heated to 90° C. The resulting PEG mixture was maintained at about 90° C. for 20 minutes. Then this PEG mixture was mixed further into the PVA solution at 90° C. (Important note: The PVA solution was made such that the 5 w/w % water that went into the PEG600 solution is accounted for, the initial PVA concentration in solution is higher than that when the bimodal PEG solution is prepared with PEG400 and PEG 200). Mixtures that contained 17.5 w/v % PVA, and 17.5 w/v % PEG600 and 17.5 w/v % PEG200; 39 w/v % PEG600 and 10 w/v % PEG200; 39 w/v % PEG600 and 17.5 w/v % PEG200; 39 w/v % PEG600 and 39 w/v % PEG200 were prepared.

Control solutions were prepared with PEG400 or PEG600 at 39 w/v %.

Alternatively, PEG600 was dissolved in PEG200, stirred vigorously, then the solution was heated to 90° C. before mixing into the PVA solution.

The resulting mixture of PVA and PEG600/PEG200 bimodal solution was not as clear (very slightly translucent) as that of a PEG 400 solution or PEG400/PEG200 bimodal solution.

For sterilization, the solution that was kept in the oven was poured into 10 cc disposable syringes (Terumo Corp, Tokyo, Japan) that were pre-heated to 90° C. They were covered with Parafilm® and packaged in vacuum (Rival Products, VS110-BCD, El Paso, Tex.). These syringes were gamma irradiated to 25 kGy (Steris, Northborough, Mass.).

The viscosity of the sterilized samples were measured by bubble tubes as described in Example 2 at 100° C.

TABLE 3
Viscosity of sterilized PVA-bimodal PEG solutions after
sterilization and reheating at 100° C. PVA concentration
was constant at 17.5 w/v % and the PVA molecular weight
was 115,000 g/mol.

| PEG | Viscosity (cP) |
|---|---|
| PEG200 (39 w/v %) | 8686 ± 253 |
| PEG400 (39 w/v %) | 8030 ± 1882 |
| PEG600 (39 w/v %) | 4789 ± 257 |
| PEG400 (39 w/v %) + PEG200 (17.5 w/v %) | 5560 ± 278 |
| PEG600 (39 w/v %) + PEG200 (17.5 w/v %) | 2733 ± 149 |

These results showed (see Table 3) that at constant PVA and PEG concentration, increasing PEG molecular weight decreased overall viscosity after sterilization. The viscosity of sterilized solutions containing bimodal concentrations of PEG was lower than single molecular weight PEG solutions despite increasing overall PEG concentration.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A cross-link-resistant and sterile injectable hydrogel formulation comprising at least one anti-cross-linking agent, wherein the anti-cross-linking agent is present during irradiation and inhibits cross-linking of the hydrogel formulation, thereby providing an irradiation cross-link-resistant and sterile injectable form of hydrogel formulation.

2. The cross-link-resistant injectable hydrogel formulation of claim 1, wherein the hydrogel is made of a vinyl polymer including poly(vinyl alcohol), poly(vinyl pyrrolidone), an acrylamide polymer including poly(N-isopropyl acrylamide), an acrylic polymer including poly(acrylic acid), poly(ethylene glycol) methacrylate, poly(ethylene-co-vinyl alcohol), a polyolefin including polyethylene, copolymers, or blends thereof.

3. The cross-link-resistant injectable hydrogel formulation of claim 1, wherein the anti-cross-linking agent is an antioxidant, a free-radical scavenger, or a combination thereof.

4. The cross-link-resistant injectable hydrogel formulation of claim 1, wherein the hydrogel comprises a polymer, polymer blends, or copolymers selected from the group consisting of polyvinyl alcohol (PVA), polyvinyl pyrrolidone (PVP), alginates, polysaccharides, poly-N-isopropyl acrylamide (PNIAAm), an acrylamide, an acrylic polymer, poly(acrylic acid), poly(ethylene glycol) methacrylate, poly(ethylene-co-vinyl alcohol), a polyolefin, a polyethylene, and combinations of two or more thereof.

5. The cross-link-resistant injectable hydrogel formulation of claim 1, wherein the hydrogel comprises a vinyl polymer, poly(vinyl pyrrolidone), an acrylamide, poly(N-isopropyl acrylamide), an acrylic polymer, poly(acrylic acid), poly(ethylene glycol) methacrylate, poly(ethylene-co-vinyl alcohol), a polyolefin, or a polyethylene, wherein one of the polymers is grafted on another polymer.

6. The cross-link-resistant injectable hydrogel formulation of claim 1, wherein the cross-linking of the hydrogel formulation during irradiation is inhibited by adding an cross-linking agent that reduces charge transfer from a solvent and by adding a second hydrophilic polymer.

7. The cross-link-resistant injectable hydrogel formulation of claim 6, wherein the second hydrophilic polymer is PEG.

8. The cross-link-resistant injectable hydrogel formulation of claim 1, wherein concentration of the anti-cross-linking agent is at least about 1000 ppm or more.

9. A method of making a cross-link-resistant and sterile injectable hydrogel formulation comprising:
a) providing a monomer, polymer or a mixture thereof in a solvent, thereby forming a hydrogel solution;
b) contacting the hydrogel solution with one or more anti-cross-linking agents, thereby forming an irradiation cross-link-resistant hydrogel solution; and
c) irradiating the cross-link-resistant hydrogel solution, thereby forming an irradiation cross-link-resistant and sterile injectable hydrogel formulation.

10. The method of claim 9 further comprising gelling the hydrogel solution prior to contacting with the anti-cross-linking agent.

11. The method of claim 10, wherein the gelling is obtained with the aid of a gellant, by chemical cross-linking, by thermal cycling, by irradiation, and/or by the application of an electric or magnetic field or a combination thereof.

12. A method of making a cross-link-resistant injectable hydrogel formulation comprising:
a) providing a monomer, polymer or a mixture thereof in a solvent, thereby forming a hydrogel solution;
b) processing the hydrogel solution to modifying at least one of its physical and/or chemical property;
c) contacting the processed hydrogel solution with one or more anti-cross-linking agents, thereby forming a cross-link-resistant hydrogel solution; and
d) irradiating the cross-link-resistant hydrogel solution, thereby forming an irradiation cross-link-resistant injectable hydrogel formulation.

13. The method of claim 12 further comprising gelling the hydrogel solution prior to contacting with the anti-cross-linking agent.

14. The method of claim 12, wherein the processing of the hydrogel solution is done by dehydration, by dehydration and annealing, by irradiation, by mechanical deformation, by the application of a magnetic or electric field, or by application of pressure.

15. A method of making a cross-link-resistant injectable hydrogel formulation comprising:
a) providing a monomer, polymer or a mixture thereof in a solvent, thereby forming a hydrogel solution;
b) adding at least one anti-cross-linking agent to the hydrogel solution, thereby forming a cross-link-resistant hydrogel solution; and
c) irradiating the hydrogel solution, thereby forming a cross-link-resistant injectable hydrogel formulation.

16. A method of inhibiting cross-linking of injectable hydrogel formulation:
a) monomer, polymer or a mixture thereof in a solvent, thereby forming a hydrogel solution;
b) adding at least one anti-cross-linking agent to the hydrogel solution, thereby forming a cross-link-resistant hydrogel solution; and
c) irradiating the cross-link-resistant hydrogel solution, thereby forming an irradiation cross-link-resistant injectable hydrogel formulation.

17. The method according to claim 15, wherein the hydrogel is made of a vinyl polymer including poly(vinyl alcohol), poly(vinyl pyrrolidone), an acrylamide polymer including poly(N-isopropyl acrylamide), an acrylic polymer including poly(acrylic acid), poly(ethylene glycol) methacrylate, poly(ethylene-co-vinyl alcohol), a polyolefin including polyethylene, copolymers, or blends thereof.

18. The method according to claim 15, wherein the anti-cross-linking agent is an antioxidant, a free-radical scavenger, or a combination thereof.

19. The method according to claim 15, wherein the injectable hydrogels are cross-linked by electron-beam radiation, gamma-radiation, beta-emitters, glutaraldehyde cross-linking, epichlorohydrin (EP) cross-linking, or by photo-initiated cross-linking.

20. The method according to claim 15, wherein the hydrogel comprises a monomer, polymer, polymer blends, or copolymers selected from the group consisting of polyvinyl alcohol (PVA), polyvinyl pyrrolidone (PVP), alginates, polysaccharides, poly-N-isopropyl acrylamide (PNIAAm), an acrylamide, an acrylic polymer, poly(acrylic acid), poly(ethylene glycol) methacrylate, poly(ethylene-co-vinyl alcohol), a polyolefin, a polyethylene, and combinations of two or more thereof.

21. The method according to claim 15, wherein the hydrogel comprises a vinyl polymer, poly(vinyl pyrrolidone), an acrylamide, poly(N-isopropyl acrylamide), an acrylic polymer, poly(acrylic acid), poly(ethylene glycol) methacrylate, poly(ethylene-co-vinyl alcohol), a polyolefin, or a polyethylene, wherein one of the polymers is grafted on another polymer.

22. The method according to claim 15, wherein the cross-linking of the hydrogel during irradiation is inhibited by adding an cross-linking agent that reduces charge transfer from a solvent and by adding a second hydrophilic polymer.

23. The method of claim 22, wherein the second hydrophilic polymer is PEG.

24. The method according to claim 15, wherein the cross-linking of the hydrogel solution during irradiation is further inhibited by using low molecular weight polymer in preparing the hydrogel solution.

25. The method according to claim 15, wherein concentration of the anti-cross-linking agent is at least about 1000 ppm or more.
